(19) United States
(12) Patent Application Publication
(10) Pub. No.: US 2020/0163854 A1
(43) Pub. Date: May 28, 2020

(54) LIFE-EXTENDING AGENT, ANTI-AGING AGENT, COSMETIC, AND FOOD/BEVERAGE COMPOSITION

(71) Applicants: KYOTO UNIVERSITY, Kyoto (JP); KYOTO PREFECTURAL PUBLIC UNIVERSITY CORPORATION, Kyoto (JP); NATIONAL UNIVERSITY CORPORATION KYOTO INSTITUTE OF TECHNOLOGY, Kyoto (JP)

(72) Inventors: Kenji SATO, Kyoto (JP); Osam MAZDA, Kyoto (JP); Tsunao KISHIDA, Kyoto (JP); Yoshihiro INOUE, Kyoto (JP)

(73) Assignees: KYOTO UNIVERSITY, Kyoto (JP); KYOTO PREFECTURAL PUBLIC UNIVERSITY CORPORATION, Kyoto (JP); NATIONAL UNIVERSITY CORPORATION KYOTO INSTITUTE OF TECHNOLOGY, Kyoto (JP)

(21) Appl. No.: 16/635,549

(22) PCT Filed: Aug. 2, 2018
(86) PCT No.: PCT/JP2018/029016

§ 371 Date: Jan. 30, 2020

(30) Foreign Application Priority Data
Aug. 2, 2017 (JP) 2017-149768

(51) Int. Cl. A61K 8/44; A61K 8/49; A23L 33/175; A61Q 19/08

(52) U.S. Cl. CPC A61K 8/44; A61Q 19/08; A23L 33/175; A61K 8/4913

(57) ABSTRACT

This invention provides a life-extending agent comprising at least one member selected from the group consisting of D-proline, D-hydroxyproline, and D-aspartic acid.

### TECHNICAL FIELD

The present invention relates to a life-extending agent, and an anti-aging agent.

The present invention also relates to a life-extending cosmetic, a life-extending food or beverage composition, an anti-aging cosmetic, and an anti-aging food or beverage composition.

### BACKGROUND ART

Recent progress in analytical technologies has revealed that various D-amino acids are present in microorganisms and plants, and also in mammals, and perform a variety of physiological functions. These findings have been attracting attention.

Patent Literature (PTL) 1 discloses hydroxyproline (or an N-acylated derivative of hydroxyproline) as a cosmetic having skin-aging inhibiting action and a skin-quality improving action.

PTL 2 discloses using the ratio of the concentration of L-form/D-form of a specific amino acid as a marker for early diagnosis of renal failure.

PTL 3 discloses a composition containing D-aspartic acid or D-alanine as a composition for promoting collagen production and improving skin conditions, such as photoaging and wrinkles.

PTL 4 discloses a cosmetic, containing hydroxyproline or an N-acyl derivative of hydroxyproline, for inhibiting skin aging and improving skin quality.

### CITATION LIST

### Patent Literature

PTL 1: JP2002-080321A
PTL 2: JP2015-132598A
PTL 3: WO2011/040363
PTL 4: JP4714071B

### SUMMARY OF INVENTION

### Technical Problem

An object of the present invention is to provide a life-extending agent, an anti-aging agent, as well as a cosmetic comprising the life-extending agent or the anti-aging agent; and a food or beverage composition comprising the life-extending agent or the anti-aging agent.

### Solution to Problem

The present invention provides the following life-extending agent, cosmetic, and food or beverage composition.

Item 1. A life-extending agent comprising at least one member selected from the group consisting of D-proline, D-hydroxyproline, and D-aspartic acid.

Item 2. A life-extending cosmetic comprising at least one member selected from the group consisting of D-proline, D-hydroxyproline, and D-aspartic acid.

Item 3. A life-extending food or beverage composition comprising at least one member selected from the group consisting of D-proline, D-hydroxyproline, and D-aspartic acid.

The present invention further provides the following anti-aging agent, cosmetic, and food or beverage composition.

Item A. An anti-aging agent comprising D-proline.
Item B. An anti-aging cosmetic comprising D-proline.
Item C. An anti-aging food or beverage composition comprising D-proline.

### Advantageous Effects of Invention

The present invention provides a life-extending agent, a cosmetic that exerts a life-extending action, and a food or beverage composition that exerts a life-extending action, the agent, cosmetic, and composition each comprising at least one member selected from the group consisting of D-proline, D-hydroxyproline, and D-aspartic acid.

Further, the present invention provides a novel anti-aging agent, a cosmetic that exerts an anti-aging action, and a food or beverage composition that exerts an anti-aging action, the agent, cosmetic, and composition each comprising D-proline.

D-proline, D-hydroxyproline, and D-aspartic acid, in particular, D-proline, can suppress the aging of T cells via the Menin-Bach2 pathway, thus suppressing the aging of the entire body.

In the present invention, D-proline is demonstrated to exert a life-extending action on animals, such as Drosophila flies. Therefore, the active ingredient of the present invention is expected to activate the functions of brain, immunity, etc., which is necessary to extend the life span. For example, the active ingredient of the present invention is expected to improve the functions deteriorated due to aging, i.e., the functions of central nerves, peripheral nerves, autonomic nerves, cardiovascular system, respiratory organs, digestive organs, hepato-biliary-pancreas, bones, cartilages, muscles, ligaments, locomotor system, skin, connective tissue, fat, brown fat, endocrine and exocrine tissues, mammary gland, renal and urinary organs, reproductive system, sensory system, pharynx, larynx, head and neck, tooth, periodontal tissue, tongue, metabolism, hematopoietic organ, or immune system. In particular, the active ingredient of the present invention is expected to exert actions, such as suppressing memory loss and suppressing a reduction in immune functions.

The aging-promoting Drosophila flies used in the Examples are a useful experimental animal; the loss of Cu/Zn-SOD causes insufficient elimination of active oxygen, showing an aging-promoting action, thus resulting in the shortening of the life span. The active ingredient of the present invention, which extends the life span, is capable of suppressing aging caused by accumulation of active oxygen and is thus also capable of suppressing the accompanying shortening of the life span; that is, the active ingredient of the present invention is capable of not only exerting a life-extending action, but also delaying aging caused by active oxygen. For example, by applying the life-extending cosmetic of the present invention to the skin, the cell life of the skin can be extended, and young skin conditions can be maintained for a long period of time. By ingesting the life-extending food or beverage composition of the present invention, the effect of active oxygen on the internal body can be reduced, the internal body can stay young, and the healthy life span can be extended.

### BRIEF DESCRIPTION OF DRAWINGS

FIG. 1 is a graph showing the lifespan-extending effect of D-proline on adult Drosophila flies in a concentration-dependent manner. Tester strain: Drosophila flies, premature aging strain, male. Feeding was performed at a concentration of 20, 60, 200, or 400 mg/ml after mixing with feed. This graph shows that D-proline dose-dependently extends the life span of aging-promoting adult Drosophila flies. L-proline shows no effect.

FIG. 2 is a graph showing the lifespan-extending effect of proline-like D-amino acids on adult Drosophila flies. Tester strain: Drosophila flies, premature aging strain, male. Feeding was performed at a concentration of 60, 200, or 400 mg/ml after mixing with feed. This graph indicates that D-aspartic acid at 400 μg/ml extends the life span of aging-promoting adult Drosophila flies (log-rank test p<0.05).

FIG. 3 is a graph showing the lifespan-extending effect of proline-like D-amino acids on adult Drosophila flies. Tester strain: Drosophila flies, premature aging strain, male. Feeding was performed at a concentration of 60, 200, or 400 mg/ml after mixing with feed. This graph indicates that D-hydroxyproline at 400 μg/ml extends the life span of aging-promoting adult Drosophila flies (log-rank test p<0.05).

FIG. 4 is a protocol diagram showing the procedures of Examples 4 to 6.

FIG. 5 is graphs showing the analytical results of CD27/CD62L expression in spleen-derived mononuclear cells (flow cytometry) (Day 495). The CD27(−)/CD62L(−) cells serve as indices of senescence.

FIG. 6 is graphs with regard to the Menin and Bach2 expression in spleen-derived mononuclear cells (real-time RT-PCR) (Day 495).

### DESCRIPTION OF EMBODIMENTS

Examples of the active ingredients of the life-extending agent of the present invention include D-proline, D-hydroxyproline, and D-aspartic acid. These ingredients can be used alone, or in a combination of two or more. These D-compounds may be pure D-compounds, or a mixture of the D-compounds and L-compounds at any ratio (including racemic mixtures).

The structural formulas of D-proline, D-hydroxyproline, and D-aspartic acid are shown below.

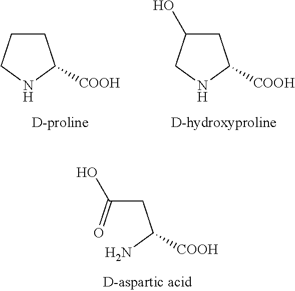 [SMILES: O=C(O)[C@H]1CCCN1 ; O=C(O)[C@H]1CC(O)CN1 ; N[C@H](CC(=O)O)C(=O)O]

Examples of the active ingredients of the anti-aging agent of the present invention include D-proline. This D-compound may be the pure D-compound, or a mixture of the D-compound and L-compound at any ratio (including racemic mixtures).

Only D-compounds serve as the active ingredients for life extension or anti-aging. L-compounds do not exert a life-extending action or anti-aging action.

The life-extending agent and anti-aging agent of the present invention, as well as a cosmetic and a food or beverage composition comprising the life-extending agent or anti-aging agent, can be applied to humans and pets, such as dogs and cats; and are preferably applied to humans.

For oral ingestion of the life-extending agent of the present invention, the amount per day for an adult is about 1 mg to 25 g, preferably about 10 mg to 20 g, and more preferably about 100 mg to 15 g. The life-extending food or beverage composition of the present invention comprises at least one member selected from the group consisting of D-proline, D-hydroxyproline, and D-aspartic acid. For oral ingestion of this composition, the amount per day for an adult is about 1 mg to 25 g, preferably about 10 mg to 20 g, and more preferably about 100 mg to 15 g.

For oral ingestion of the anti-aging agent of the present invention, the amount per day for an adult is about 1 mg to about 25 g, preferably about 10 mg to about 20 g, and more preferably about 100 mg to about 15 g. The anti-aging food or beverage composition of the present invention comprises D-proline. For oral ingestion of this composition, the amount per day for an adult is about 1 mg to 25 g, preferably about 10 mg to 20 g, and more preferably about 100 mg to 15 g.

For application of the life-extending agent of the present invention to humans as a medicine or an external skin preparation of a cosmetic, the concentration of the at least one member selected from the group consisting of D-proline, D-hydroxyproline, and D-aspartic acid is about 0.001 to 10% by mass, and preferably about 0.01 to 5% by mass.

For application of the anti-aging agent of the present invention to humans as a medicine or an external skin preparation of a cosmetic, the concentration of D-proline is about 0.001 to 10% by mass, and preferably about 0.01 to 5% by mass.

The life-extending agent and anti-aging agent of the present invention can be ingested as pharmaceutical preparations. Examples of the formulations include tablets, capsules, granules, powders, pills, troches, liquids, injections, drops, inhalants, drinks, syrups, suppositories, transdermal absorbents, patches, sachets, plasters, ointments, and the like.

The cosmetic of the present invention include foundations, emulsions, lotions, lotions, lipsticks, blushers, makeup bases, and the like.

Examples of foods and beverages include milk drinks, fermented milk drinks, carbonated drinks, fruit juice drinks, soft drinks, sports drinks, supplemental nutrition drinks, and other drinks, candy, candy, gum, chocolate, tablet confections, snacks, biscuits, jellies, jams, cream, baked confections, ice cream, yogurt, butter, breads, supplements, dietary supplements, liquid foods, and the like.

### EXAMPLES

Hereinafter, the present invention is described in more detail with reference to Examples. However, the present invention is obviously not limited to these Examples.

### Example 1

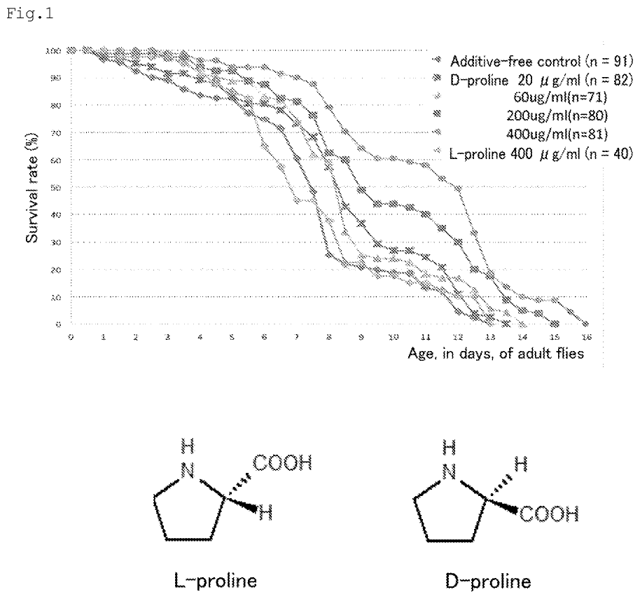

Mutants of adult Drosophila flies (male) containing Cu/Zn-SOD with reduced activity due to amino acid substitution (sod1[n1]) were bred in a plastic tube having a diameter of 25 mm, and fed with a simple medium (instant medium) for Drosophila that contains D-proline (D-Pro) at a concentration of 20, 60, 200, or 400 μg/ml, or L-proline (L-Pro) at a concentration of 400 μg/ml, followed by measuring the life span of the flies. FIG. 1 shows the results, together with the number (n) of Drosophila melanogaster flies used in the test. For the control, a simple medium to which no D-Pro or L-Pro was added was used.

As shown in FIG. 1, the life span of the Drosophila flies increased in a concentration-dependent manner.

### Example 2

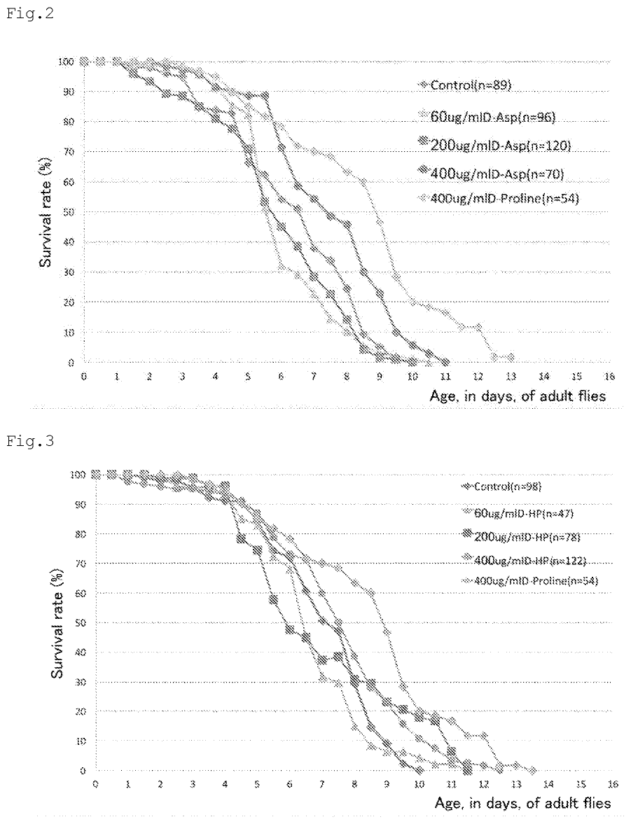

Mutants of adult Drosophila flies (male) containing Cu/Zn-SOD with reduced activity due to amino acid substitution (sod1[n1]) were bred in a plastic tube having a diameter of 25 mm, and fed with a simple medium (instant medium) for Drosophila that contains D-proline (D-Pro) at a concentration of 400 μg/ml, or D-aspartic acid (D-Asp) at a concentration of 60, 200, or 400 μg/ml, followed by measuring the life span of the flies. FIG. 2 shows the results, together with the number (n) of Drosophila melanogaster flies used in the test. For the control, a simple medium to which no D-Pro or D-Asp was added was used.

As shown in FIG. 2, D-Asp extended the life span of Drosophila flies in a concentration-dependent manner, but D-Pro showed a significantly higher effect.

### Example 3

Mutants of adult Drosophila flies (male) containing Cu/Zn-SOD with reduced activity due to amino acid substitution (sod1[n1]) were bred in a plastic tube having a diameter of 25 mm, and fed with a simple medium (instant medium) for Drosophila that contains D-proline (D-Pro) at a concentration of 400 μg/ml, or D-hydroxyproline (D-HP) at a concentration of 60, 200, or 400 μg/ml, followed by measuring the life span of the flies. FIG. 3 shows the results, together with the number (n) of Drosophila melanogaster flies used in the test. For the control, a simple medium to which no D-Pro or D-HP was added was used.

As shown in FIG. 3, D-HP extended the life span of Drosophila flies in a concentration-dependent manner, but D-Pro showed a significantly higher effect.

### Example 4

Normal water (control group; N=3), water in which L-proline (produced by Nacalai Tesque, Inc.) was dissolved at a concentration of 0.2 mg/ml (L-proline group; N3), or water in which D-proline (produced by Nacalai Tesque Inc.) was dissolved at a concentration of 0.2 mg/ml (D-proline group; N3) was administered to C57BL/6 mice (8 weeks old) (purchased from Shimizu Laboratory Supplies Co., Ltd.) by free water intake. After euthanasia on Day 495, the spleen was removed and ground through a 0.45-μM strainer to prepare a suspension of spleen-derived cells (FIG. 4). After centrifugation at 1000 g for 5 minutes, the supernatant was removed by suction. Thereafter, 2 ml of ammonium chloride hemolytic agent (distilled water containing 8.99 mg/ml of NH₄Cl, 1 mg/ml of KHCO₃, and 37 μg/ml of EDTA4Na) was added to the cell pellets, followed by suspension. The resulting product was then allowed to stand at room temperature for 2 minutes. Subsequently, 10 ml of RPMI 1640 medium with 10% FBS was added thereto, the mixture was centrifuged at 1000 g for 5 minutes, and the supernatant was removed by suction. Then, 5 ml of RPMI 1640 medium with 10% FBS was added thereto to prepare a cell suspension (spleen-derived mononuclear cells). This cell suspension was used in Examples 5 and 6.

### Example 5

After placing 1×10⁶ spleen-derived mononuclear cells of Example 4 into a 1.5-ml Eppendorf tube, centrifugation was performed at 900 g for 5 minutes. Then, the supernatant was removed by suction, and 50 μl of FACS buffer (PBS(−) in which 0.5% BSA, 0.01% NaNO, and 1 mM EDTA were dissolved) was added thereto. Thereafter, incubation was performed on ice for 20 minutes with an FITC-labeled mouse anti-CD27 antibody (NOVUS; NPB-1-44021) and a PE-labeled mouse anti-CD62L antibody (ABGENT; ATB10190). After washing twice with an FACS buffer, analysis was performed with flow cytometry (Becton Dickinson, FACSCalibur).

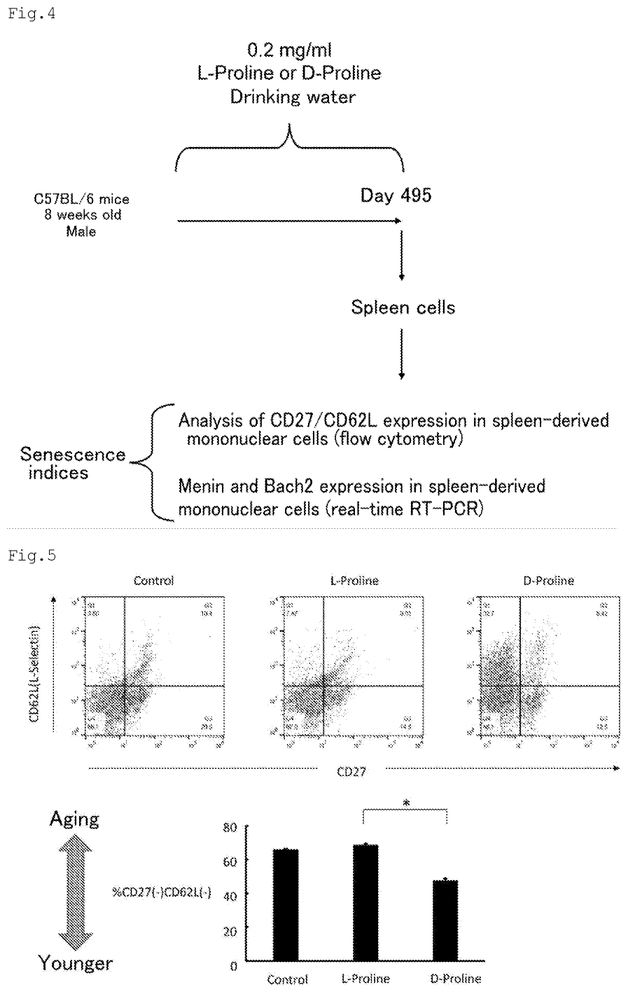

FIG. 5 shows the results. In the D-proline administration group, a significantly reduced number of T cells with a senescence marker of CD62-negative or CD27-negative were found, compared to the control group or the L-proline administration group. This suggests that the administration of D-proline contributed to suppression of aging of the immune system.

### Example 6

Total RNA was extracted from 1×10⁵ spleen-derived mononuclear cells of Example 4 using an RNeasy Mini Kit, produced by Qiagen. Then, cDNA was synthesized from this RNA using ReverTra Ace qPCR RT Master Mix, produced by Toyobo Co., Ltd. This cDNA was mixed with Real-Time PCR Master Mix, primers specific to the Menin gene, Bach2 gene, or β-actin gene, and a TaqMan probe. qRT-PCR was performed using an AB 7300 Real-Time PCR System, produced by ABI. The mRNA levels of the Menin gene and the Bach2 gene were quantified as a ratio relative to the β-actin gene mRNA level, and calculated taking the value of the spleen-derived mononuclear cells of the control group as 1.

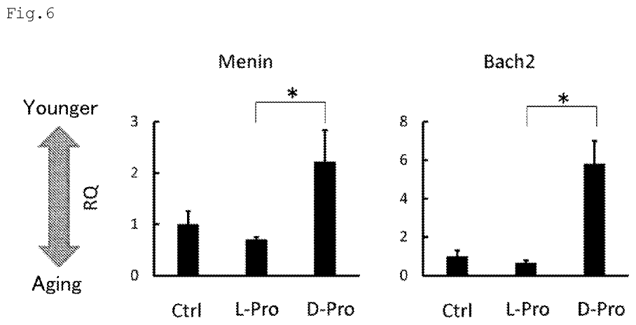

FIG. 6 shows the results. In the D-proline administration group, a higher mRNA expression of the Menin gene and Batch gene was seen, compared to the control group or the L-proline administration group. This suggests that the Menin-Bach2 pathway is involved in suppressing T-cell senescence by D-proline administration.

1.-3. (canceled)

4. A method for suppressing aging, comprising applying a composition comprising D-proline to a subject in need thereof.

5. The method according to claim 4, wherein the composition is a cosmetic composition.

6. The method according to claim 4, wherein the composition is applied to skin.

7. A method for suppressing aging, comprising administrating a composition comprising D-proline to a subject in need thereof.

8. The method according to claim 7, wherein the composition is a food or beverage composition.

9. A method for extending life span, comprising applying a composition comprising at least one member selected from the group consisting of D-proline and D-hydroxyproline to a subject in need thereof.

10. The method according to claim 9, wherein the composition comprises D-proline.

11. The method according to claim 9, wherein the composition is a cosmetic composition.

12. The method according to claim 9, wherein the composition is applied to skin.

13. A method for extending life span, comprising administrating a composition comprising at least one member selected from the group consisting of D-proline and D-hydroxyproline to a subject in need thereof.

14. The method according to claim 13, wherein the composition comprises D-proline.

15. The method according to claim 13, wherein the composition is a food or beverage composition.
